(19) United States
(12) Patent Application Publication
(10) Pub. No.: US 2014/0375785 A1
(43) Pub. Date: Dec. 25, 2014

(54) IMAGING-BASED MONITORING OF STRESS AND FATIGUE

(71) Applicant: Raytheon Company, Waltham, MA (US)

(72) Inventors: John A. Kogut, Bowie, MD (US); Marc Berte, Ashburn, VA (US)

(21) Appl. No.: 13/921,310

(22) Filed: Jun. 19, 2013

(51) Int. Cl. A61B 5/18; A61B 5/0205; A61B 5/00; A61B 5/11; G06K 9/00; H04N 5/33

(52) U.S. Cl. CPC A61B 5/18; G06K 9/00268; G06K 9/0061; H04N 5/33; A61B 5/0077; A61B 5/1103; A61B 5/1128; A61B 5/0075; A61B 5/0205; A61B 5/0082; A61B 5/02405
USPC 348/77; 600/476; 600/473; 600/479

(57) ABSTRACT

An example system can monitor the stress and fatigue of a subject. The system can include a light source configured to direct illuminating light onto a face of the subject. The illuminating light can reflect off the face of the subject to form reflected light. The system can include collection optics that collect a portion of the reflected light and produce video-rate images of the face of the subject. The system can include an image processor configured to locate an eye in the video-rate images, extract fatigue signatures from the located eye, and determine a fatigue level of the subject, in part, from the fatigue signatures. The image processor can also be configured to locate a facial region away from the eye in the video-rate images, extract stress signatures from the located facial region, and determine a stress level of the subject from the stress signatures.

### TECHNICAL FIELD

Embodiments pertain to monitoring of stress and fatigue of a subject. Such monitoring is suitable for use with vehicle drivers, air traffic controllers, pilots of aircraft and of remotely piloted vehicles, and other suitable stressful occupations.

### BACKGROUND

There are many stressful occupations in which an operator performs a particular task for an extended period of time. For instance, vehicle drivers, air traffic controllers, pilots of aircraft and of remotely piloted vehicles, and operators of power plant and computer network systems all require extended periods of concentration from the respective operators. For many of these occupations, a lapse in concentration could result in death, injury, and/or damage to equipment. Such a lapse in concentration can be caused by an elevated level of fatigue and/or an elevated level of stress for the operator.

### SUMMARY

Many current monitoring systems rely on contact with a subject. For instance, measurement of heart rate and/or heart rate variability can use an optical finger cuff, such as the type used for pulse oximetry, an arm cuff with pressure sensors, such as the type used for blood pressure measurement, and/or electrodes, such as the type used for electrocardiography. In many cases, use of a device that contacts the subject can be uncomfortable or impractical. There exists a need for a monitoring system that can operate at a distance from a subject, without contacting the subject.

An example monitoring system, discussed below, can extract both fatigue and stress information from video images of a face of a subject. More specifically, fatigue and stress information can be collected simultaneously from a single optical system. Advantageously, the monitoring system does not use direct contact with the subject. In some examples, the monitoring system may operate from a distance of about 1 meter, or a range of about 0.5 meters to about 1.5 meters.

The fatigue information can be extracted from behavior of one or both eyes of the subject. For instance, an erratic eye behavior gaze, an increasing or unusual number of eye blinks, and/or an increasing or unusual number of eye closures can indicate an increasing or high level of fatigue of the subject. In addition, an increasing or unusual number of yawns and/or micronods can also indicate an increasing or high level of fatigue of the subject. The yawns and/or micronods can be measured from one or more portions of the face other than the eyes.

The stress information can be extracted from one or more regions of the face of the subject, away from the eyes of the subject, such as the forehead or cheeks of the face. For example, an increasing or unusual heart rate, an increasing or unusual heart rate variability, and/or an increasing or unusual respiration rate can indicate an increasing or high level of stress of the subject. Increasing and/or high levels of fatigue and/or stress can be used to trigger one or more further actions, such as providing a warning, such as to a system operator or a system controller, and/or triggering an alert to the subject.

In some examples, the face of the subject is illuminated with infrared light. The infrared light is invisible to the subject, and is not disruptive to the subject, so that the monitoring system can be used in a dark environment. The collection optics in the monitoring system can include a spectral filter that blocks most or all of the light outside a particular wavelength range. In some examples, the spectral filter can block most or all of the visible portion of the spectrum, so that the monitoring system can be used in the presence of daylight and ambient light without degrading in performance.

An example system can monitor the stress and fatigue of a subject. The system can include collection optics that collect a portion of the light reflected from a face of the subject and produce video-rate images of the face of the subject. The system can include an image processor configured to locate an eye in the video-rate images, extract fatigue signatures from the located eye, and determine a fatigue level of the subject, in part, from the fatigue signatures. The image processor can also be configured to locate a facial region away from the eye in the video-rate images, extract stress signatures from the located facial region, and determine a stress level of the subject from the stress signatures.

Another example system can monitor the stress and fatigue of a subject. The system can include a light source configured to direct illuminating light onto a face of the subject. The light source can include at least one infrared light emitting diode. The illuminating light can have a spectrum that includes a first wavelength. The illuminating light can reflect off the face of the subject to form reflected light. The system can include collection optics that collect a portion of the reflected light and produce video-rate images of the face of the subject at the first wavelength. The collection optics and the light source can be spaced apart from the subject by a distance between 0.5 meters and 1.5 meters. The collection optics can include a spectral filter that transmits wavelengths in a wavelength band that includes the first wavelength and blocks wavelengths outside the transmitted wavelength band. The collection optics can include a lens configured to form an image of the face of the subject. The collection optics can include a detector configured to detect the image of the face of the subject at the first wavelength. The system can include an image processor configured to locate an eye in the video-rate images, extract fatigue signatures from the located eye, the fatigue signatures comprising at least one of eye behavior gaze, eye blinks, and eye closure rate, and determine a fatigue level of the subject, in part, from the fatigue signatures. The image processor can also be configured to locate a facial region away from the eye in the video-rate images, extract stress signatures from the located facial region, the stress signatures comprising at least one of heart rate, heart rate variability, and respiration rate, and determine a stress level of the subject from the stress signatures.

An example method can monitor the stress and fatigue of a subject. Video-rate images of a face of the subject can be received. An eye can be located in the video-rate images. Fatigue signatures can be extracted from the located eye. A fatigue level of the subject can be determined, in part, from the fatigue signatures. A facial region away from the eye can be located in the video-rate images. Stress signatures can be extracted from the located facial region. A stress level of the subject can be determined from the stress signatures.

This summary is intended to provide an overview of subject matter of the present patent application. It is not intended to provide an exclusive or exhaustive explanation of the invention. The Detailed Description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic drawing of an example system for monitoring the stress and fatigue of a subject.

FIG. 2 is a schematic drawing of an example configuration of collection optics for the system of FIG. 1.

FIG. 3 is a schematic drawing of another example configuration of collection optics for the system of FIG. 1.

FIG. 4 is a schematic drawing of another example configuration of collection optics for the system of FIG. 1.

FIG. 5 is a plan drawing of an example video image, with examples of the eyes located in the video image and an example facial area located away from the eyes.

FIG. 6 is a schematic drawing of an example computer/image processor detecting various fatigue signatures and stress signatures and determining a fatigue level and a stress level.

FIG. 7 is a perspective drawing of an example monitoring system, as mounted in the steering wheel of an automobile.

FIG. 8 is a flow chart of an example method of operation for the monitoring system of FIG. 1.

### DETAILED DESCRIPTION

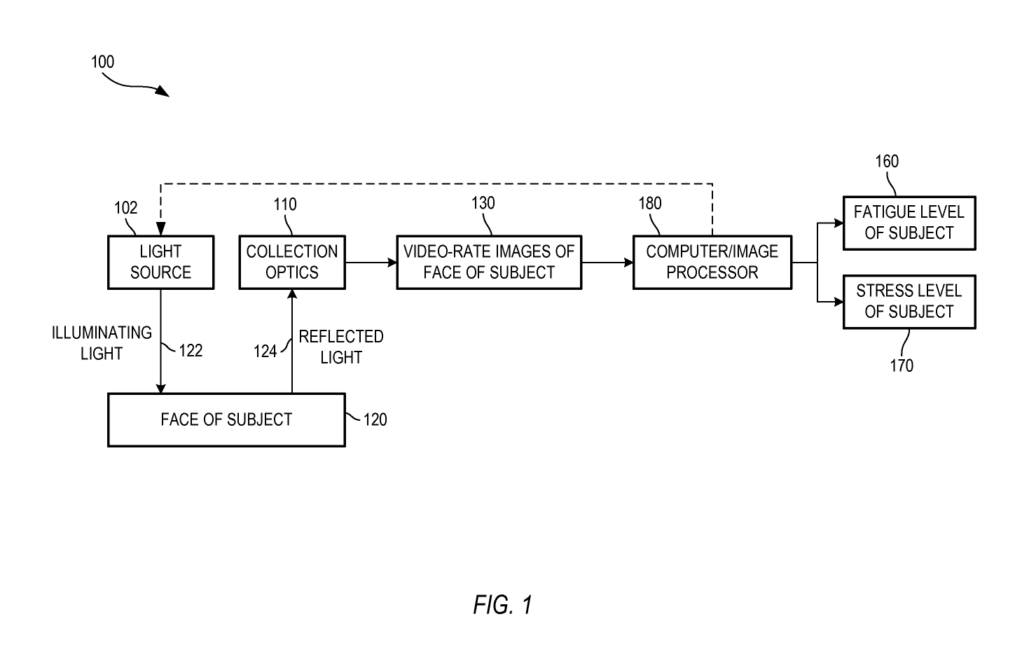

FIG. 1 is a schematic drawing of an example system 100 for monitoring the stress and fatigue of a subject. A light source 102 illuminates a face 120 of the subject. Collection optics 110 collect light reflected off the face 120 of the subject and form a series of video-rate images 130 of the face 120 of the subject. A computer and/or image processor 180 extracts one or more fatigue signatures and one or more stress signatures from the video-rate images 130. The fatigue signatures can determine a fatigue level 160 of the subject. The stress signatures can determine a stress level 170 of the subject. Each of these elements or groups of elements is discussed in more detail below.

The light source 102 produces illuminating light 122. The light source 102 is located near an expected location of the subject, so that when the subject is present, the illuminating light 122 strikes the face 120 of the subject. For instance, if the system 100 is mounted in an automobile, then the light source 102 may be mounted in the dashboard or on the steering wheel, and may direct the illuminating light 122 toward an expected location for a driver's face. The illuminating light 122 can diverge from the light source 102 with a cone angle sized to fully illuminate the face 120 of the subject, including a tolerance on the size and placement of the face 120 of the subject. In some cases, there may be more than one light source, and the light sources may be located away from each other. For instance, an automobile may include light sources above the door, above the windshield, in the dashboard, and in other suitable locations. In these examples, each light source directs illuminating light toward an expected location of the face of the subject. In some examples, the optical path can include a diffuser between the light source and the expected location of the subject.

The visible portion of the electromagnetic spectrum extends from wavelengths of 400 nm to 700 nm. The infrared portion of the electromagnetic spectrum extends from wavelengths of 700 nm to 1 mm. In some examples, the illuminating light 122 includes at least one spectral component in the infrared portion of the spectrum, with no light in the visible portion of the spectrum, so that the illuminating light 122 is invisible to the subject. In other examples, the illuminating light 122 includes at least one spectral component in the infrared portion of the spectrum and at least one spectral component in the visible portion of the spectrum. In some examples, the illuminating light 122 includes only one spectral component; in other examples, the illuminating light 122 includes more than one spectral component. Examples of suitable light sources 102 can include a single infrared light emitting diode, a plurality of infrared light emitting diodes that all emit light at the same wavelength, a plurality of infrared light emitting diodes where at least two light emitting diodes emit light at different wavelengths, and a plurality of light emitting diodes where at least one emits in the infrared portion of the spectrum and at least one emits in the visible portion of the spectrum.

For light emitting diodes, the spectral distribution of the light output can be characterized by a center wavelength and a spectral width. In some examples, the illuminating light 122 has a center wavelength in the range of 750 nm to 900 nm, in the range of 800 nm to 850 nm, in the range of 750 nm to 850 nm, and/or in the range of 800 nm to 900 nm. In some examples, the illuminating light 122 has a spectral width less than 50 nm, less than 40 nm, less than 30 nm, and/or less than 20 nm.

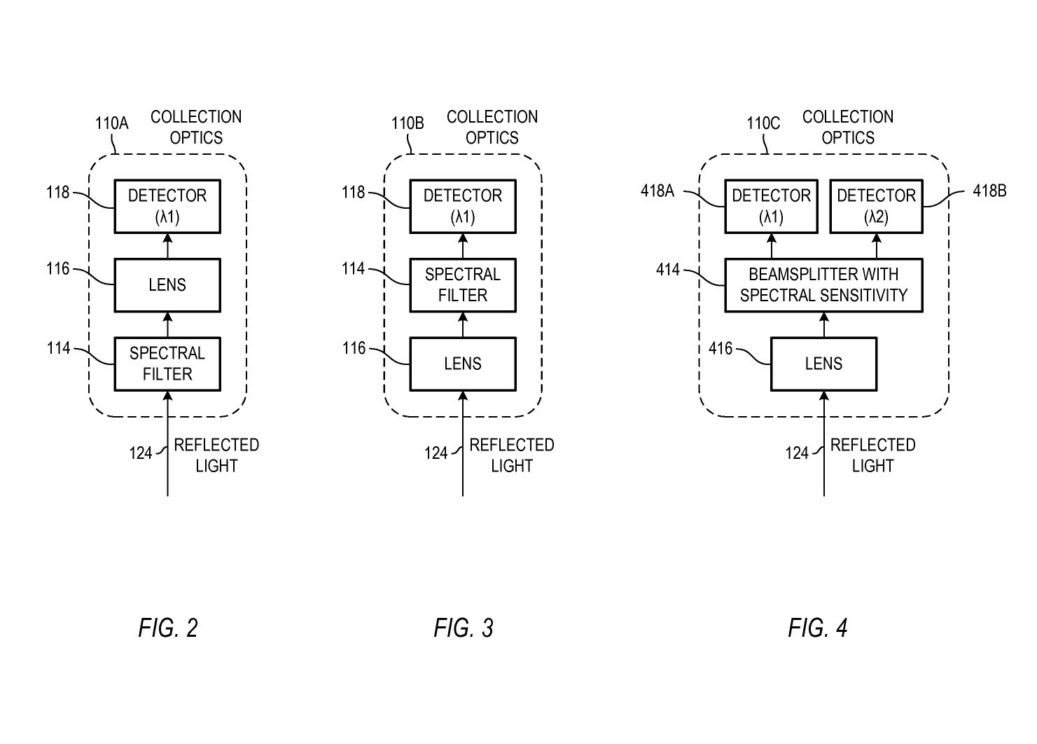

The illuminating light 122 reflects off the face 120 of the subject to form reflected light 124. The collection optics 110 collect a portion of the reflected light 124 and produce video-rate images 130 of the face 120 of the subject. The illuminating light 122 can have a spectrum that includes a first wavelength, denoted as λ1 in FIG. 1. The collection optics 110 can produce the video-rate images 130 at the first wavelength. Three example configurations for the collection optics 110 are shown in FIGS. 2-4, and are discussed below in detail. In some examples, the collection optics 110 can be packaged with the light source 102 in a common housing. The common housing can be located in a suitable location, such as on the dashboard or steering wheel of an automobile.

In some examples, a computer and/or image processor 180 can control the light source 102 and can receive the video-rate 130 images of the face 120 of the subject. The computer can include at least one processor, memory, and a machine-readable medium for holding instructions that are configured for operation with the processor and memory. An image processor may be included within the computer, or may be external to the computer.

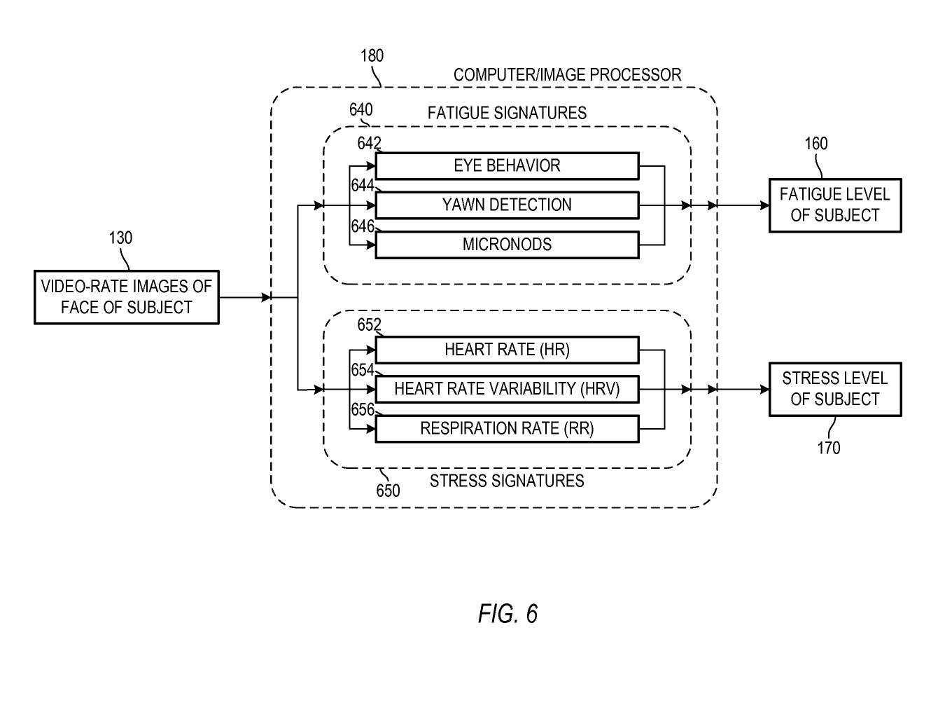

The image processor 180 can process the video-rate images 130. For instance, the image processor can sense the location of various features, such as eyes, in the video-rate images 130, can determine a gaze direction for the eyes of the subject, can sense when the subject yawns or undergoes a micronod, and can sense heart rate, heart rate variability, and respiration rate from the video-rate images 130. In addition to processing the video-rate images 130 in real time, the computer can also maintain a recent history of properties, so that the computer can sense when a particular property changes. The computer can also maintain baseline or normal ranges for particular quantities, so that the computer can sense when a particular quantity exits a normal range. The computer can perform weighting between or among various signatures to determine an overall fatigue or stress level. Various fatigue signatures and stress signatures are shown in FIG. 6, and are discussed below in more detail.

FIG. 2 is a schematic drawing of an example configuration of collection optics 110A for the system 100 of FIG. 1. The collection optics 110A receive reflected light 124 that is generated by the light source 102 and reflects off the face 120 of the subject. If the light source 102 produces illuminating light 122 having a spectrum that includes a first wavelength, then the collection optics 110A can produce the video-rate images 130 at the first wavelength.

The collection optics 110A can include a spectral filter 114 that transmits wavelengths in a wavelength band that includes the first wavelength and blocks wavelengths outside the transmitted wavelength band. Suitable spectral filters 114 can include, but are not limited to, edge filters and notch filters.

In some examples, the first wavelength is in the infrared portion of the spectrum. For these examples, the spectral filter 114 can block most or all ambient light or daylight. As such, the video-rate images 130 are formed with light having a spectrum that corresponds to that of the light source 102. In addition, the video-rate images 130 have an intensity that is relatively immune to the presence of daylight or ambient light, which is desirable.

The collection optics 110A can include a lens 116 configured to form an image of the face 120 of the subject. Light received into the collection optics 110A passes through the spectral filter 114, and is focused by the lens 116 to form an image. When the subject is present, the image formed by the lens 116 is of the face 120 of the subject.

The collection optics 110A can include a detector 118 configured to detect the image of the face 120 of the subject at the first wavelength. The first wavelength is denoted as λ1 in FIG. 2. When the subject is present, the collection optics 110 can image the face 120 of the subject onto the detector 118. Suitable detectors 118 can include, but are not limited to, CCD or CMOS video sensors. The detector 118 can produce video-rate images 130 of the face 120 of the subject. Suitable video frame rates can include, but are not limited to, 10 Hz, 12 Hz, 14 Hz, 16 Hz, 18 Hz, 20 Hz, 22 Hz, 24 Hz, 25 Hz, 26 Hz, 28 Hz, 30 Hz, 36 Hz, 48 Hz, 50 Hz, 60 Hz, or more than 60 Hz.

FIG. 3 is a schematic drawing of another example configuration of collection optics 110B for the system 100 of FIG. 1. In this configuration, the spectral filter 114 is disposed between the lens 116 and the detector 118. While both configurations 110A, 110B can produce video-rate images 130 of the face 120 of the subject at the first wavelength, there may be instances when one configuration can be advantageous over the other for reasons unrelated to optical performance. For instance, if the collection optics are packaged in a housing, and the housing includes a transparent cover, then in some cases, it may be desirable to attach the spectral filter to the transparent cover, or to use the spectral filter as the transparent cover itself. For these examples, the configuration of FIG. 2 may be preferable. In other examples, the spectral filter 114 may be incorporated onto a front surface of the detector 118, or may be included on a cover glass that is disposed in front of the detector. For these examples, the configuration of FIG. 3 may be preferable.

The collimation optics 110A, 110B of FIGS. 2 and 3 can be used with light sources 102 that emit light at a single wavelength. As an alternative, FIG. 4 shows an example configuration of collection optics 110C that can be used with light sources 102 that emit light at two different wavelengths. If the light source 102 produces illuminating light 122 having a spectrum that includes first and second wavelengths, then the collection optics 110C can produce video-rate images 130 at the first wavelength, and can also produce video-rate images 130 at the second wavelength.

The collimation optics 110C can include a spectrally-sensitive beamsplitter 414 that transmits wavelengths in a first wavelength band that includes the first wavelength, λ1, and reflects wavelengths in a second wavelength band that includes the second wavelength, λ2. The collimation optics 110C can include a lens 416 configured to form a first image of the face 120 of the subject at the first wavelength and a second image of the face 120 of the subject at the second wavelength. In practice, the lens 416 may be similar in structure and function to the lens 116, with the beamsplitter 414 disposed in the optical path after the lens 416. The beamsplitter 414 can direct a first optical path, at the first wavelength, onto a first detector 418A. The beamsplitter 414 can direct a second optical path, at the second wavelength, onto a second detector 418B. The first detector 418A can be configured to detect the image of the face of the subject at the first wavelength. The second detector 418B can be configured to detect the image of the face of the subject at the second wavelength.

The collection optics 110C can produce two sets of video-rate images 130, with one set at the first wavelength and the other set at the second wavelength. In some examples, the image processor 180 can be configured to locate the eye in one of the first and second video-rate images 130 and locate the facial region in the other of the first and second video-rate images 130. In some examples, the first and second wavelengths are in the infrared portion of the spectrum. In other examples, one of the first and second wavelengths is in the infrared portion of the spectrum, and the other of the first and second wavelengths is in the visible portion of the spectrum.

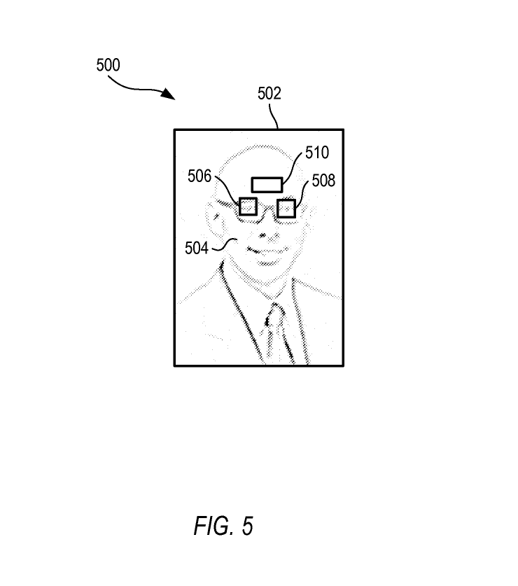

FIG. 5 is a plan drawing of an example video image 500, which is one image from the stream of video-rate images 130. The image processor 180 can search within the boundary 502 of the image 500, can determine whether a face 504 is present in the image 500, can automatically locate one or both eyes 506, 508 in the face 504, and can automatically locate at least one other region 510 in the face away from the eyes 506, 508. The other region 510 may be a location on a forehead or on the cheeks of the face. From the located regions on the face, such as 506, 508, 510, the image processor 180 can record various signatures that can be linked with a fatigue level or a stress level for the subject.

FIG. 6 is a schematic drawing of an example computer/image processor 180 detecting various fatigue signatures 640 and stress signatures 650, and determining a fatigue level 160 and a stress level 170 from the respective signatures. The image processor 180 receives the video-rate images 130 of the face 120 of the subject. The video-rate images 130 can be a single stream of images at a single wavelength, or can include two streams of images at different wavelengths.

The fatigue signatures 640 include one or more of eye behavior 642, yawn detection 644, and micronods 646. The eye behavior 642 can be extracted from one or both eyes in the video-rate images 130. The eye behavior 642 can include one or more of eye gaze, eye blinks, and eye closure rate. Yawn detection 644 may include the mouth of the face in the video-rate images 130. Micronods 646, such as the small jerking of the head when the subject is nodding off, can be extracted from the position of the face, as well as one or both eyes.

For each of the fatigue signatures 640, the computer can establish a baseline or “normal” range of operation. For instance, the eye blinks may be measured in blinks per minute, and normal range can extend from a low value of blinks per minute to a high value of blinks per minute. The normal range can be determined by a history of past behavior from the subject, and can therefore vary from subject-to-subject. Alternatively, the normal range can be predetermined, and can be the same for all subjects.

When the subject becomes fatigued, the subject may blink more often. This increased rate of blinking may extend beyond the high value in the normal range. Alternatively, the rate of blinking may have a rate of increase that exceeds a particular threshold, such as more than 10% within a minute, or another suitable value and time interval. This departure from the normal range of operation can provide the computer with an indication that the subject may be fatigued or may be becoming fatigued.

The eye blinking can be just one indicator of fatigue. The yawn detection 644 and micronods 646 may have similar normal ranges, and may provide the computer with indications of fatigue when the sensed values are outside the normal ranges. The computer can use data from the eye behavior 642, yawn detection 644, and micronods 646 singly or in any combination, in order to determine a level of fatigue. The fatigue level 160 determined by the computer can have discrete values, such as “normal”, “mildly fatigued”, and “severely fatigued”. Alternatively, the fatigue level 160 can have a value on a continuous scale, where specified values or ranges on the continuous scale can indicate that the subject is “normal”, “mildly fatigued”, or “severely fatigued”.

The stress signatures 650 include one or more of heart rate (HR) 652, heart rate variability (HRV) 654, and respiration rate (RR) 656. The stress signatures 650 can be extracted from one or more regions away from the eyes in the video-rate images 130, such as on the forehead or one or both cheeks. Each stress signature can have its own normal range of operation, and can provide the computer with an indication when the signature behavior moves outside the normal range of operation. The information from the stress signatures 650 can be taken singly or combined in any combination to determine a stress level 170 of the subject. The stress level may have discrete values, or may alternatively use a continuum.

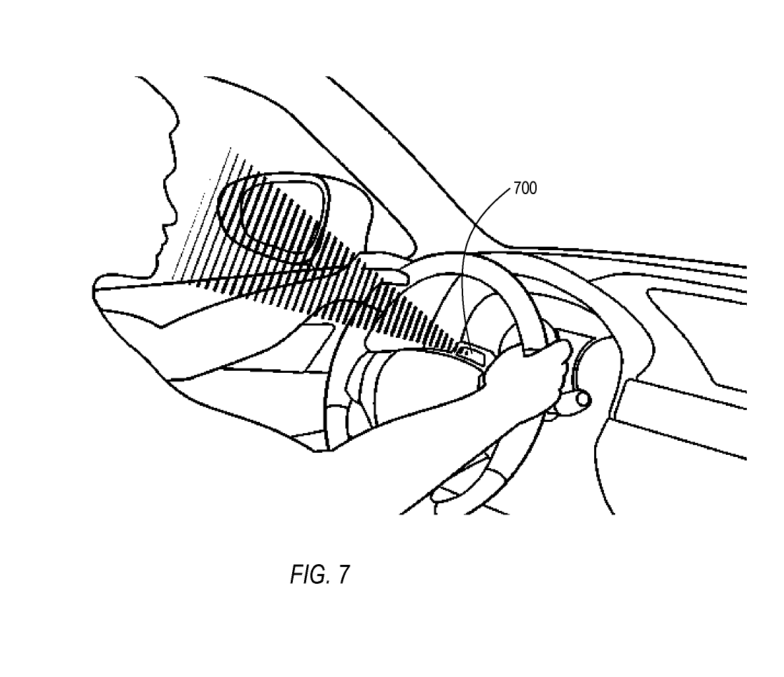

FIG. 7 is a perspective drawing of an example monitoring system 700, as mounted in the steering wheel of an automobile. The light source in the sample directs illuminating light, in the infrared portion of the spectrum, onto the face of the subject. Light reflects off the face of the subject. A portion of the reflected light is collected by the collection optics, which are also mounted in the steering wheel near the light source. The computer/image processor may be located with the light source and collection optics, may be located elsewhere in the automobile, or may be located at an external location. The video-rate images may be transmitted from the detector to the image processor by hard wiring, by wireless connection within the automobile, or by wireless connection that uses an external network, such as a cellular telephone network.

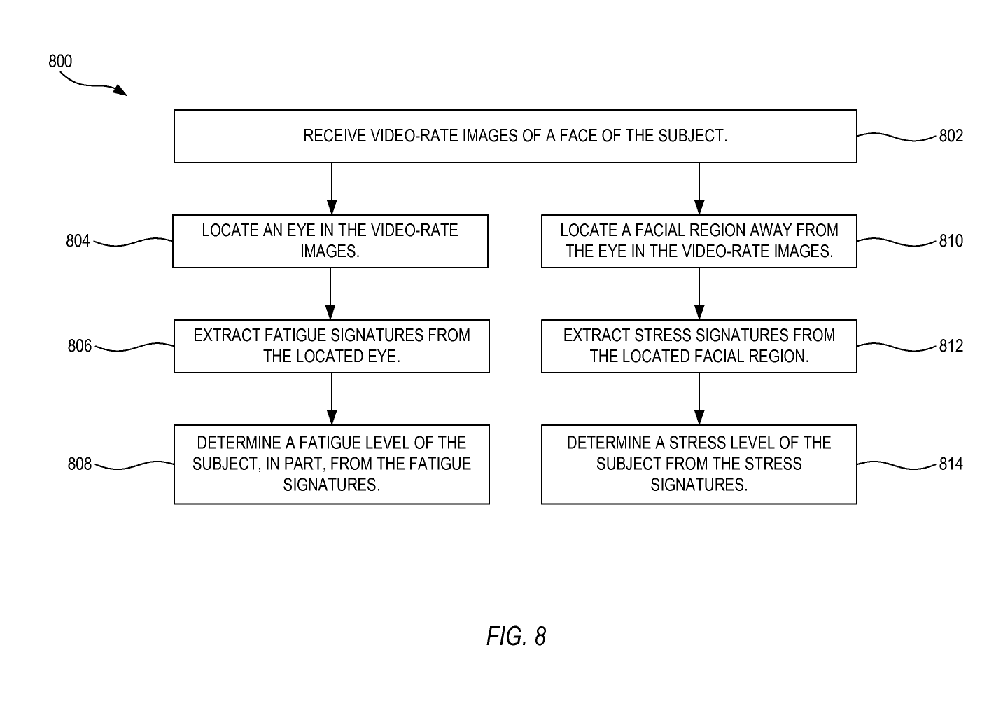

FIG. 8 is a flow chart of an example method of operation 800 for monitoring stress and fatigue of a subject. The method of operation 800 can be executed using the monitoring system 100 of FIG. 1, or with another monitoring system. Step 802 receives video-rate images of a face of the subject, such as the video-rate images 130 of the face 120 of the subject as shown in FIG. 1. Step 804 locates an eye in the video-rate images, such as eye 506 or eye 508 as shown in FIG. 5. Step 806 extracts fatigue signatures from the located eye, such as fatigue signatures 640 as shown in FIG. 6. Step 808 determines a fatigue level of the subject, in part, from the fatigue signatures, such as fatigue level 160 as shown in FIG. 1. Step 810 locates a facial region away from the eye in the video-rate images, such as region 510 in FIG. 5. Step 812 extracts stress signatures from the located facial region, such as stress signatures 650 as shown in FIG. 6. Step 814 determines a stress level of the subject from the stress signatures, such as stress level 170 as shown in FIG. 1. Steps 804-808 may be performed before, after, or interleaved with steps 810-814.

An additional step can include directing illuminating light onto a face of the subject, where the illuminating light reflects off the face of the subject to form reflected light. Another additional step can include collecting a portion of the reflected light. Another additional step can include producing the video-rate images from the collected light.

Some embodiments may be implemented in one or a combination of hardware, firmware and software. Embodiments may also be implemented as instructions stored on a computer-readable storage device, which may be read and executed by at least one processor to perform the operations described herein. A computer-readable storage device may include any non-transitory mechanism for storing information in a form readable by a machine (e.g., a computer). For example, a computer-readable storage device may include read-only memory (ROM), random-access memory (RAM), magnetic disk storage media, optical storage media, flash-memory devices, and other storage devices and media. In some embodiments, system 100 may include one or more processors and may be configured with instructions stored on a computer-readable storage device.

What is claimed is:

1. A system for monitoring stress and fatigue of a subject, the system comprising:
collection optics that collect a portion of light reflected from a face of the subject and produce video-rate images of the face of the subject; and
an image processor configured to:
locate an eye in the video-rate images;
extract fatigue signatures from the located eye;
determine a fatigue level of the subject, in part, from the fatigue signatures;
locate a facial region away from the eye in the video-rate images;
extract stress signatures from the located facial region; and
determine a stress level of the subject from the stress signatures.

2. The system of claim 1, further comprising a light source configured to direct illuminating light onto the face of the subject, the illuminating light reflecting off the face of the subject to form the reflected light.

3. The system of claim 2,
wherein the illuminating light has a spectrum that includes a first wavelength; and
wherein the collection optics produce the video-rate images at the first wavelength.

4. The system of claim 3, wherein the collection optics comprise:
a spectral filter that transmits wavelengths in a wavelength band that includes the first wavelength and blocks wavelengths outside the transmitted wavelength band;
a lens configured to form an image of the face of the subject; and
a detector configured to detect the image of the face of the subject at the first wavelength.

5. The system of claim 3, wherein the first wavelength is in the infrared portion of the spectrum.

6. The system of claim 2,
wherein the illuminating light has a spectrum that includes first and second wavelengths; and
wherein the collection optics produce first video-rate images at the first wavelength and produce second video-rate images at the second wavelength.

7. The system of claim 6, wherein the collection optics comprise:
a spectrally-sensitive beamsplitter that transmits wavelengths in a first wavelength band that includes the first wavelength, and reflects wavelengths in a second wavelength band that includes the second wavelength;
a lens configured to form a first image of the face of the subject at the first wavelength and a second image of the face of the subject at the second wavelength;
a first detector configured to detect the image of the face of the subject at the first wavelength; and
a second detector configured to detect the image of the face of the subject at the second wavelength.

8. The system of claim 7, wherein the image processor is configured to locate the eye in one of the first and second video-rate images and locate the facial region in the other of the first and second video-rate images.

9. The system of claim 6, wherein the first and second wavelengths are in the infrared portion of the spectrum.

10. The system of claim 6, wherein one of the first and second wavelengths is in the infrared portion of the spectrum, and the other of the first and second wavelengths is in the visible portion of the spectrum.

11. The system of claim 1, wherein the fatigue signatures comprise at least one of eye gaze, eye blinks, and eye closure rate.

12. The system of claim 11, wherein the fatigue signatures further comprise at least one of yawn detection and micronods, the at least one of yawn detection and micronods being extracted from the video-rate images.

13. The system of claim 1, wherein the stress signatures comprise at least one of heart rate (HR), heart rate variability (HRV), and respiration rate (RR).

14. The system of claim 1, wherein the light source comprises a plurality of light-emitting diodes, at least two of the light-emitting diodes producing light having different emission spectra.

15. The system of claim 1, wherein the light source and the collection optics are spaced apart from the subject by a distance between 0.5 meters and 1.5 meters.

16. A system for monitoring stress and fatigue of a subject, the system comprising:
a light source configured to direct illuminating light onto a face of the subject, the light source comprising at least one infrared light emitting diode, the illuminating light having a spectrum that includes a first wavelength, the illuminating light reflecting off the face of the subject to form reflected light;
collection optics that collect a portion of the reflected light and produce video-rate images of the face of the subject at the first wavelength, the collection optics comprising:
a spectral filter that transmits wavelengths in a wavelength band that includes the first wavelength and blocks wavelengths outside the transmitted wavelength band;
a lens configured to form an image of the face of the subject; and
a detector configured to detect the image of the face of the subject at the first wavelength; and
an image processor configured to:
locate an eye in the video-rate images;
extract fatigue signatures from the located eye, the fatigue signatures comprising at least one of eye behavior gaze, eye blinks, and eye closure rate;
determine a fatigue level of the subject, in part, from the fatigue signatures;
locate a facial region away from the eye in the video-rate images;
extract stress signatures from the located facial region, the stress signatures comprising at least one of heart rate (HR), heart rate variability (HRV), and respiration rate (RR); and
determine a stress level of the subject from the stress signatures.

17. The system of claim 16, wherein the collection optics and the light source are spaced apart from the subject by a distance between 0.5 meters and 1.5 meters.

18. A method for monitoring stress and fatigue of a subject, the method comprising:
receiving video-rate images of a face of the subject;
locating an eye in the video-rate images;
extracting fatigue signatures from the located eye;
determining a fatigue level of the subject, in part, from the fatigue signatures;
locating a facial region away from the eye in the video-rate images;
extracting stress signatures from the located facial region; and
determining a stress level of the subject from the stress signatures.

19. The method of claim 18, further comprising:
directing illuminating light onto a face of the subject, the illuminating light reflecting off the face of the subject to form reflected light;
collecting a portion of the reflected light; and
producing the video-rate images from the collected light.

20. The method of claim 18,
wherein the fatigue signatures comprise at least one of eye behavior gaze, eye blinks, and eye closure rate; and
wherein the stress signatures comprise at least one of heart rate (HR), heart rate variability (HRV), and respiration rate (RR).
